(19) United States
(12) Patent Application Publication
(10) Pub. No.: US 2026/0217657 A1
(43) Pub. Date: Jul. 30, 2026

(54) PYRAZOLIDINE-3, 5-DIONE BASED COMPOUNDS AND A PROCESS FOR PREPARATION THEREOF

(71) Applicant: COUNCIL OF SCIENTIFIC & INDUSTRIAL RESEARCH, New Delhi (IN)

(72) Inventors: Santosh Baburao MHASKE, Pune (IN); Priyanka HALDER, Pune (IN)

(21) Appl. No.: 19/147,555

(22) PCT Filed: Jan. 11, 2024
(86) PCT No.: PCT/IN2024/050029

§ 371 Date: Jul. 11, 2025

(30) Foreign Application Priority Data
Jan. 11, 2023 (IN) 202311002669

(51) Int. Cl. C07D 231/54; C07D 231/34; C07D 231/36

(52) U.S. Cl. CPC C07D 231/54; C07D 231/34; C07D 231/36

(57) ABSTRACT

The present invention relates to 5,6-diaryl-5,6-diazaspiro [2.4]heptane-4,7-dione compound of formula (I) and the process of preparation thereof. The present invention also relates to the compound of formula (I) and its use in the preparation of sulfinpyrazone uricosuric drug or its derivatives/analogues. The compound of formula (I) is useful as anti-malarial, anti-microbial, anti-bacterial, anti-inflammatory, COX-2 inhibition, and anti-analgesics agents.

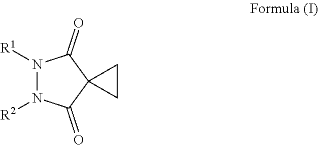

### FIELD OF THE INVENTION

Present invention relates to a 5,6-diaryl-5,6-diazaspiro[2.4]heptane-4,7-dione compound of formula

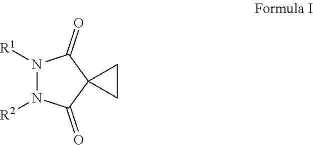

Particularly, present invention relates to a process for the preparation of compound of formula I. More particularly, present invention relates to the compound of formula (I) useful in the preparation of sulfinpyrazone uricosuric drug and/or its derivatives/analogues.

### BACKGROUND OF THE INVENTION

Sulfinpyrazone, a derivative of phenylbutazone, and its intermediate G-25671 exhibits potent anti-uricosuric activity by reducing the concentration of uric acid in blood. Sulfinpyrazone, chemical name is 1,2-diphenyl-4-(2-(phenylsulfinyl) ethyl) pyrazolidine-3,5-dione. It is mostly inhibiting the urate anion transporter, which is responsible for the reabsorption of urate in proximal convoluted tubules. It can also stop platelet aggregation by inhibiting COX and increase platelet survival time and treat ischemic cardiovascular and cerebrovascular diseases. It shows weak anti-inflammatory, analgesic effects and prevents gouty arthritis. In 1959, it was approved by the US Food and Drug Administration (FDA), and later marketed by Novartis as the Anturane brand name.

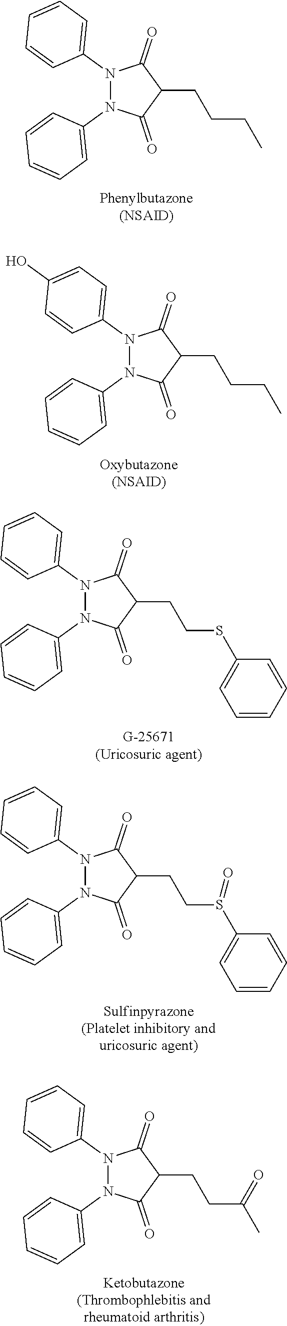 [SMILES: CCCCC1C(=O)N(c2ccccc2)N(c2ccccc2)C1=O ; CCCCC1C(=O)N(c2ccccc2)N(c2ccc(O)cc2)C1=O ; O=C1C(CCSc2ccccc2)C(=O)N(c2ccccc2)N1c1ccccc1 ; O=C1C(CCS(=O)c2ccccc2)C(=O)N(c2ccccc2)N1c1ccccc1 ; CC(=O)CCC1C(=O)N(c2ccccc2)N(c2ccccc2)C1=O]

### Derivatives of Phenylbutazone

Functionalized pyrazolidine-3,5-diones (C₃H₄N₂O₂) feature a novel class of heterocyclic compounds that possess a diverse biological activity including anti-microbial, anti-bacterial, anti-inflammatory, COX-2 inhibition, anti-analgesics, as well as material application. The construction of pyrazolidine-3,5-diones has been attracting increased attention because of its immense diversity in the biological as well as pharmaceutical field.

In the last few decades, a lot of research has been done on the functionalized pyrazolidine-3,5-diones to reveal its biological activity and it was prepared by the traditional condensation reaction between the derivatives of malonic ester and diphenylhydrazine, which was developed by Emil Fischer in the late 19th century. However, the use of diphenylhydrazine substrate inevitably leads to environmental and health concerns; exhibits major drawbacks towards the synthesis of sulfinpyrazone. The carcinogenic nature of hydrazine building blocks requires extra safety arrangements for handling and that makes a big problem for up-scaling in industry. Furthermore, the cost of the diphenylhydrazine is very high (44,200/100 gm in TCI) and only the simple hydrazine molecule is available in the market.

Thus, the generation of libraries of these drug congeners relies on the ominous synthetic route with very low yield and efficiency. In order to address this issue, development of an economical and eco-friendly synthetic route has always been of great interest in the scientific field. Therefore, the sulfinpyrazone drug and its potent intermediate G-25671 featuring the pyrazolidine-3,5-dione core and possessing uricosuric activity caught our attention. To date, few synthetic routes have resulted in the successful process development of this drug and they involve the use of carcinogenic and expensive diphenylhydrazine as a starting material for the formation of pyrazolidine-3,5-diones core. To overcome this disadvantage and to provide a general access to pyrazolidine-3,5-diones, we designed a novel metal-free synthetic strategy to construct the pyrazolidine-3,5-diones core via N—N bond formation. We envisioned that the presence of functionalizable cyclopropyl moiety would also facilitate the N—N bond formation by exerting ring strain.

Nitrogen-nitrogen bond, particularly in the cyclic compounds, is an omnipresent structural framework in numerous bioactive natural products, drugs, dyes, and organic materials. Complementarily, in the last few decades, several remarkable methods have been developed to form intermolecular as well as intramolecular N—N bond via various pathways and sources. By the reason of high electronegativity of nitrogen atoms and the nucleophilic nature of N—H functional group, retrosynthetic disconnection of N—N bond has always been an infrequently targeted way and its formation becomes more challenging for dehydrogenative N—N coupling reactions. Although, the cross dehydrogenative coupling reactions have reformed the area of orthodox organic transformations by providing an excellent efficiency, step and atom economy; however, a precisely designed oxidizing system is required to activate the particular N—H bond by avoiding undesired C—C and C—N Coupled side products. Therefore, a contemporary interest has been devoted to developing a specific strategy for the formation of N—N bond in cyclic compounds particularly in pyrazolidine-3,5-diones for its enormous bioactivity. The use of simple and easily accessible dianilide precursors for the construction of pyrazolidine-3,5-diones via intramolecular N—N bond formation was not achieved until a significantly revolutionized work was reported by T. Gieshoff D. Schollmeyer and S R. Waldvogel, Angew. Chem., Int. Ed., 2016, 55, 9437, to access pyrazolidine-3,5-dione through electrochemical anodic N—N bond formation using undivided cell. Of late, copper catalyzed intramolecular N—N bond formation to access pyrazolidine-3,5-dione was developed by Y.-H. Liu, H. Song, C. Zhang, Y.-J. Liu and B.-F. Shi, Chin. J. Chem., 2020, 38, 1545; however, this method was limited to single substrate scope and very low yield.

Undoubtedly, organic electrosynthesis is an environmentally benign process; but it has its own advantages and shortcomings especially at commercial scale. In the modem economy model, decreasing the production and manufacturing cost for the drug synthesis in large scale is becoming a major concern and that encouraged us to develop intramolecular dehydrogenative N—N bond formation as a key-step in the construction of the pyrazolidine-3,5-dione core of sulfinpyrazone class of drugs.

In literature, a conventional known method for synthesis of dione compound produces very toxic byproducts, cumbersome to separate and scale up, and harmful to environment, thus, rendering the process not preferable. Therefore, there is an unmet need in the art to provide a highly efficient method for synthesis of pyrazolidine-3,5-dione core of sulfinpyrazone drug via N—N bond formation using easily accessible and biologically relevant precursors under very mild reaction conditions.

### Objective of the Invention

Main objective of the present invention is to provide a 5,6-diaryl-5,6-diazaspiro [2.4]heptane-4,7-dione compounds of formula (I).

Another objective of the present invention is to provide a process for synthesis of 5,6-diaryl-5,6-diazaspiro [2.4]heptane-4,7-dione compound of formula (I) from diamide precursors.

Yet another object of the present invention is to provide a compound of formula (I) that is synthesized by metal free oxidative intramolecular dehydrogenative N—N bond formation via hypervalent iodine mediated reaction of dianilide precursors providing the pyrazolidine-3,5-dione core of the sulfinpyrazone class of drugs under mild reaction conditions.

Yet another object of the present invention is to provide a compound of formula (I) that is used in the preparation of sulfinpyrazone uricosuric agent and/or its derivatives/analogues.

Yet another object of the present invention is to provide a process for synthesis of compound of formula (I) which is less toxic, eco-friendly, cheaper, high yielding, safe and simple, has good chemical selectivity and it is easy to realize industrial production.

### SUMMARY OF THE INVENTION

The present invention provides a 5,6-diaryl-5,6-diazaspiro [2.4]heptane-4,7-dione compound of formula (I) and a process of preparation thereof. The present invention also relates to the compound of formula (I) and its use in the preparation of sulfinpyrazone uricosuric drugs and/or its derivatives/analogues.

Accordingly, present invention provides a 5,6-diaryl-5,6-diazaspiro [2.4]heptane-4,7-dione compound of formula (I)

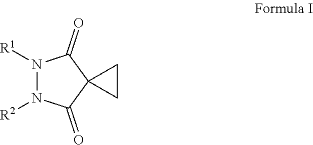

wherein
R¹ and R² may be same or different, having substituents selected from the group consisting of hydrogen, substituted or unsubstituted (C₆-C₁₀)aryl, substituted or unsubstituted (C₁-C₆)alkyl, substituted or unsubstituted heterocyclyl, —(CH₂)-substituted or unsubstituted (C₆-C₁₀)aryl, substituted or unsubstituted (C₃-C₁₀)alkene, substituted or unsubstituted (C₃-C₁₀)alkyne, ferrocene, and substituted or unsubstituted (C₁-C₆)alkoxy.

In an embodiment of the present invention, the compound of formula (I) is selected from the group consisting of.
i. 5,6-di-p-tolyl-5,6-diazaspiro[2.4]heptane-4,7-dione;
ii. 5,6-di-m-tolyl-5,6-diazaspiro[2.4]heptane-4,7-dione;
iii. 5,6-bis(3,4-dimethylphenyl)-5,6-diazaspiro[2.4]heptane-4,7-dione;
iv. 5,6-bis(3,5-dimethylphenyl)-5,6-diazaspiro[2.4]heptane-4,7-dione;
v. 5,6-bis(4-isopropylphenyl)-5,6-diazaspiro[2.4]heptane-4,7-dione;
vi. 5,6-bis(4-(tert-butyl)phenyl)-5,6-diazaspiro[2.4]heptane-4,7-dione;
vii. 5,6-bis(4-iodophenyl)-5,6-diazaspiro[2.4]heptane-4,7-dione;
viii. 5,6-bis(4-bromophenyl)-5,6-diazaspiro[2.4]heptane-4,7-dione;
ix. 5,6-bis(4-chlorophenyl)-5,6-diazaspiro[2.4]heptane-4,7-dione;
x. 5,6-bis(3-chlorophenyl)-5,6-diazaspiro[2.4]heptane-4,7-dione;
xi. 5,6-bis(3,4-dichlorophenyl)-5,6-diazaspiro[2.4]heptane-4,7-dione;
xii. 5,6-bis(4-fluorophenyl)-5,6-diazaspiro[2.4]heptane-4,7-dione;
xiii. 5,6-bis(4-acetylphenyl)-5,6-diazaspiro[2.4]heptane-4,7-dione;
xiv. 5,6-bis(4-methoxyphenyl)-5,6-diazaspiro[2.4]heptane-4,7-dione;
xv. 5,6-Bis(4-ethoxyphenyl)-5,6-diazaspiro[2.4]heptane-4,7-dione;
xvi. 5,6-bis(3,4-dimethoxyphenyl)-5,6-diazaspiro[2.4]heptane-4,7-dione;
xvii. 5-(4-Bromophenyl)-6-(p-tolyl)-5,6-diazaspiro[2.4]heptane-4,7-dione; and
xviii. 4-(6-(4-Bromophenyl)-4,7-dioxo-5,6-diazaspiro[2.4]heptan-5-yl)phenyl trifluoromethanesulfonate.

In another embodiment, present invention provides a process of preparation of 5,6-diaryl-5,6-diazaspiro [2.4]heptane-4,7-dione compound of formula (I) comprising the steps of.
(a) reacting a compound of formula (3) with thionyl chloride under reflux to obtain an unstable dichloride compound of formula (3′), which was further reacted with aromatic/aliphatic amine compound of formula R¹/R²—NH₂ in presence of a base and a solvent to obtain diamide compound of formula (4); and

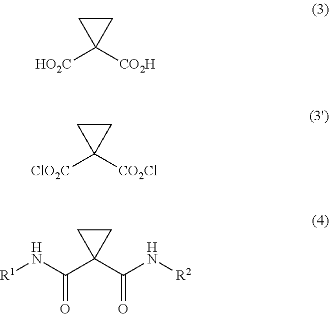

wherein R¹ and R² are the same as defined above;
(b) reacting the compound of formula (4) with an oxidant in presence of a solvent under stirring to obtain a compound of formula (I).

In yet another embodiment of the present invention, the solvent used in step (a) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), 1,2-Dichloroethane (DCE), tert-Butyl alcohol (tBuOH), acetonitrile (ACN), toluene, heptane, hexane, dichloromethane (DCM), acetic acid (AcOH) or a mixture thereof.

In yet another embodiment of the present invention, the base used in step (a) is selected from the group consisting of triethylamine (Et₃N), potassium carbonate, potassium bicarbonate, sodium carbonate, sodium bicarbonate, potassium hydroxide, and sodium hydroxide.

In yet another embodiment of the present invention, the oxidant used in step (b) is selected from the group consisting of diacetoxyiodobenzene (PIDA), Phenyliodine bis(trifluoroacetate) (PIFA) or a mixture thereof.

In yet another embodiment of the present invention, the solvent used in step (b) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), dichloroethane (DCE), tert-butyl alcohol (tBuOH), acetonitrile (ACN), toluene, heptane, hexane, dichloromethane (DCM), acetic acid (AcOH) or a mixture thereof.

In yet another embodiment, present invention provides a process of preparation of uricosuric drug sulfinpyrazone of Formula II using compound of formula I comprising the steps of:
a) reacting the compound of formula (I) as claimed in claim 1, with thiophenol (PhSH) as a nucleophile in the presence of sodium methoxide solution (NaOMe) and a solvent at room temperature in the range of 20 to 35° C. for 2 hours to obtain the compound of formula (IIa);

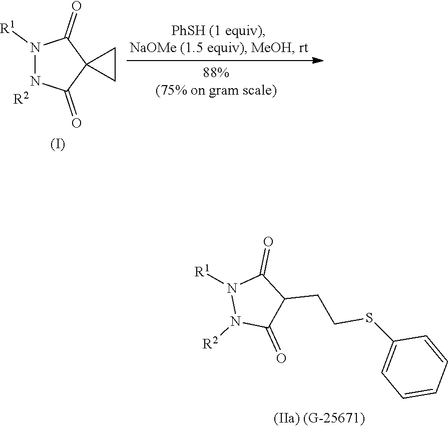

b) oxidising the compound of formula (IIa) with methanesulfonic acid (MsOH) and tert-butyl hydroperoxide (tBuOOH) in presence of a solvent at 40° C. to obtain the compound of formula (II).

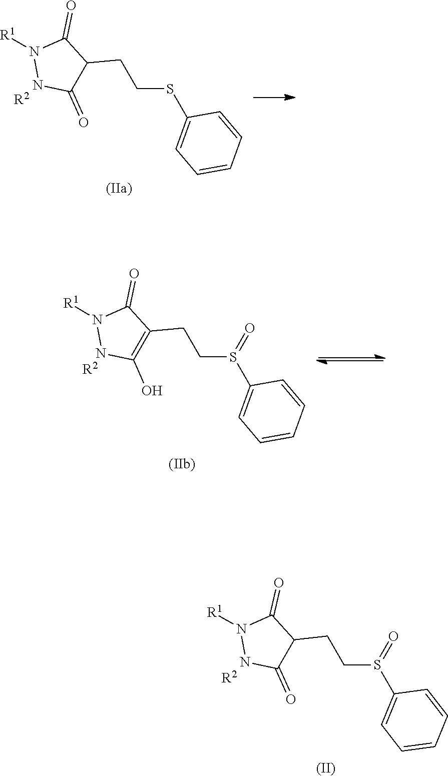

In yet another embodiment of the present invention, the solvent used in step (a) is selected from the group consisting of alcoholic solvent, THF, ACN or a mixture thereof.

In yet another embodiment of the present invention, the solvent used in step (b) is selected from the group consisting of ACN, THF, alcoholic solvent, or a mixture thereof.

In yet another embodiment of the present invention, compound of formula (I) can be also used for the preparation of other sulfinpyrazone type drug molecules and/or its derivatives/analogues such as phenylbutazone (NSAID), oxybutazone (NSAID), ketobutazone (thrombophlebitis and rheumatoid arthritis) etc. and so on.

In another embodiment of the present invention, a pharmaceutical composition comprising compound(s) of formula I and pharmaceutically acceptable excipients for prevention and/or treatment of disease(s) or disorder(s) or symptom(s). The disease(s) or disorder(s) or symptom(s) includes but not limited to, malaria, microbial infection, bacterial infection, inflammatory conditions, thrombophlebitis and rheumatoid arthritis, and so on.

In another embodiment of the present invention, the compound of formula (I) can be used in the treatment of malaria, microbial infection, bacterial infection, inflammatory conditions, thrombophlebitis and rheumatoid arthritis.

In another embodiment of the present invention, the sulfinpyrazone, and its derivatives, salts and precursor sulfide forms thereof can be used as antimalarial or antifungal agents.

### DETAILED DESCRIPTION OF THE INVENTION

The term, “(C₁₋₆) alkyl”, as used herein, refers to the radical of saturated aliphatic groups, including straight or branched-chain alkyl groups having six or fewer carbon atoms in its backbone, for instance, C₁-C₆ for straight chain and C₃-C₆ for branched chain. As used herein, (C₁₋₆) alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term “(C₃-C₁₀) alkene” and/or “(C₃-C₁₀)alkyne” as used herein, refers to the same definition as provided for the term “(C₁₋₆)alkyl” except the number of carbons and accordingly examples varies from C₃-C₁₀.

The term, “(C₁₋₆)alkoxy” refers to a (C₁₋₆)alkyl having an oxygen radical attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

The term “(C₆₋₁₀)aryl” or “aryl” as used herein refers to monocyclic or bicyclic hydrocarbon groups having 6 to 10 ring carbon atoms, wherein at least one carbocyclic ring is having a π electron system. Examples of (C₆-C₁₀) aryl ring systems include, but are not limited to, phenyl and naphthyl. Unless indicated otherwise, aryl group can be unsubstituted or substituted with one or more substituents, for example 1-4 substituents independently selected from the group consisting of halogen, (C₁₋₆)alkyl, hydroxy, cyano, nitro, —COOH, amino, acetyl, and (C₁₋₆)alkoxy.

The term, (C₅₋₁₀)heterocyclyl, as used herein refers to a 5- to 10-membered, saturated, partially unsaturated or unsaturated monocyclic or bicyclic ring system containing 1 to 4 heteroatoms independently selected from the group consisting of oxygen, nitrogen and sulfur. Saturated heterocyclic ring systems do not contain any double bond, whereas partially unsaturated heterocyclic ring systems contain at least one double bond, and unsaturated heterocyclic ring systems form an aromatic system containing heteroatom(s). The oxidized form of the ring nitrogen and sulfur atom contained in the heterocyclyl to provide the corresponding N-oxide, S-oxide or S,S-dioxide is also encompassed in the scope of the present invention. Representative examples of heterocycles include, but are not limited to, tetrahydrofuran, tetrahydrothiophene, pyrrolidine, dihydropyran, tetrahydropyran, thio-dihydropyran, thio-tetrahydropyran, piperidine, piperazine, morpholine, 1,3-oxazinane, 1,3-thiazinane, 4,5,6-tetrahydropyrimidine, 2,3-dihydrofuran, dihydrothiene, dihydropyridine, tetrahydropyridine, isoxazolidine, pyrazolidine, furan, pyrrole, thiophene, imidazole, oxazole, thiazole, triazole, tetrazole, benzofuran, indole, benzoxazole, benzothiazole, isoxazole, triazine, purine, pyridine, pyrazine, quinoline, isoquinoline, phenazine, oxadiazole, pteridine, pyridazine, quinazoline, pyrimidine, isothiazole, benzopyrazine and tetrazole. Unless stated otherwise, (C₄₋₁₀)heterocyclyl can be unsubstituted or substituted with one or more substituents, for example, substituents independently selected from the group consisting of oxo, halogen, hydroxy, cyano, nitro, amine, (C₁₋₆)alkyl and COOH.

The term, “halogen” as used herein refers to chlorine, fluorine, bromine or iodine atoms.

The present invention relates to a 5,6-diaryl-5,6-diazaspiro [2.4]heptane-4,7-dione compound of formula (I),

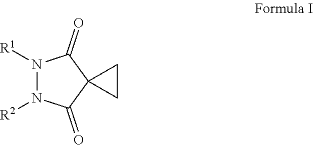

wherein
R¹ and R² may be same or different, having substituents selected from hydrogen, substituted or unsubstituted (C₆-C₁₀)aryl, substituted or unsubstituted (C₁-C₆)alkyl, substituted or unsubstituted heterocyclyl, —(CH₂)-substituted or unsubstituted (C₆-C₁₀)aryl, substituted or unsubstituted (C₃-C₁₀)alkene, substituted or unsubstituted (C₃-C₁₀)alkyne, ferrocene, and substituted or unsubstituted (C₁-C₆)alkoxy.

The 5,6-diaryl-5,6-diazaspiro [2.4]heptane-4,7-dione compound of formula (I) is selected from the group consisting of.
5,6-di-p-tolyl-5,6-diazaspiro[2.4]heptane-4,7-dione;
5,6-di-m-tolyl-5,6-diazaspiro[2.4]heptane-4,7-dione;
5,6-bis(3,4-dimethylphenyl)-5,6-diazaspiro[2.4]heptane-4,7-dione;
5,6-bis(3,5-dimethylphenyl)-5,6-diazaspiro[2.4]heptane-4,7-dione;
5,6-bis(4-isopropylphenyl)-5,6-diazaspiro[2.4]heptane-4,7-dione;
5,6-bis(4-(tert-butyl)phenyl)-5,6-diazaspiro[2.4]heptane-4,7-dione;
5,6-bis(4-iodophenyl)-5,6-diazaspiro[2.4]heptane-4,7-dione;
5,6-bis(4-bromophenyl)-5,6-diazaspiro[2.4]heptane-4,7-dione;
5,6-bis(4-chlorophenyl)-5,6-diazaspiro[2.4]heptane-4,7-dione;
5,6-bis(3-chlorophenyl)-5,6-diazaspiro[2.4]heptane-4,7-dione;
5,6-bis(3,4-dichlorophenyl)-5,6-diazaspiro[2.4]heptane-4,7-dione;
5,6-bis(4-fluorophenyl)-5,6-diazaspiro[2.4]heptane-4,7-dione;
5,6-bis(4-acetylphenyl)-5,6-diazaspiro[2.4]heptane-4,7-dione;
5,6-bis(4-methoxyphenyl)-5,6-diazaspiro[2.4]heptane-4,7-dione;
5,6-Bis(4-ethoxyphenyl)-5,6-diazaspiro[2.4]heptane-4,7-dione;
5,6-bis(3,4-dimethoxyphenyl)-5,6-diazaspiro[2.4]heptane-4,7-dione;
5-(4-Bromophenyl)-6-(p-tolyl)-5,6-diazaspiro[2.4]heptane-4,7-dione; and
4-(6-(4-Bromophenyl)-4,7-dioxo-5,6-diazaspiro[2.4]heptan-5-yl)phenyl trifluoromethanesulfonate.

In some embodiments, when the R¹ of formula I is hydrogen, then R² is not a substituted or unsubstituted (C₆-C₁₀)aryl.

In some embodiments, when the R¹ is a substituted or unsubstituted (C₁-C₆)alkyl, then R² is not a substituted or unsubstituted (C₁-C₆)alkyl.

The present invention relates to a process of preparation of 5,6-diaryl-5,6-diazaspiro [2.4]heptane-4,7-dione compound of formula (I) comprising the steps of.
(a) reacting the compound of formula (3) with thionyl chloride under reflux to obtain an unstable dichloride compound of formula (3′), which was further reacted with aromatic/aliphatic amine compound of formula R¹/R²—NH₂ (R¹ and R² are as defined above) in presence of base and solvent to obtain diamide compound of formula (4); and

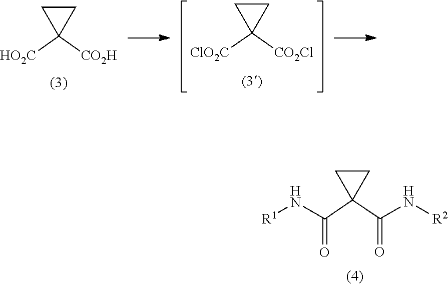

(b) reacting the compound of formula (4) with an oxidant in presence of a solvent under stirring to obtain a compound of formula (I)

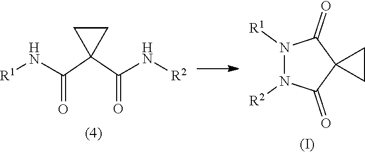

The solvent used in step (a) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), 1,2-Dichloroethane (DCE), tert-Butyl alcohol (tBuOH), acetonitrile (ACN), toluene, heptane, hexane, dichloromethane (DCM), acetic acid (AcOH) or a mixture thereof.

The base used in step (a) is selected from the group consisting of triethylamine (Et₃N), potassium carbonate, potassium bicarbonate, sodium carbonate, sodium bicarbonate, potassium hydroxide, and sodium hydroxide.

The oxidant used in step (b) is selected from the group consisting of diacetoxyiodobenzene (PIDA), Phenyliodine bis(trifluoroacetate) (PIFA) or a mixture thereof.

The solvent used in step (b) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), dichloroethane (DCE), tert-butyl alcohol (tBuOH), acetonitrile (ACN), toluene, heptane, hexane, dichloromethane (DCM), acetic acid (AcOH) or a mixture thereof.

The compound of formula (I) can have diverse biological activity including but not limited to anti-malarial, anti-microbial, anti-bacterial, anti-inflammatory, COX-2 inhibition, and anti-analgesics.

The present invention relates to a process of preparation of uricosuric drug sulfinpyrazone comprising the steps of:
a) reacting the compound of formula (I) with thiophenol (PhSH) as a nucleophile in the presence of sodium methoxide solution (NaOMe), and solvent at room temperature for 2 hours to obtain the compound of formula (IIa);

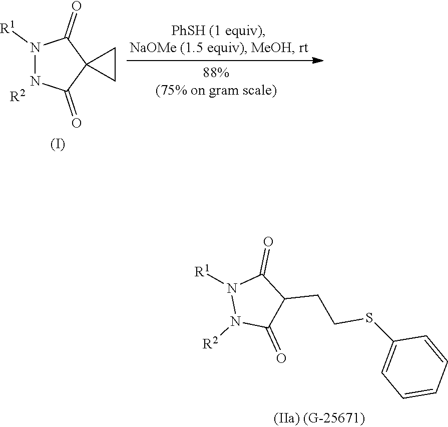

b) oxidizing the compound of formula (IIa) with methanesulfonic acid (MsOH), and tert-butyl hydroperoxide (tBuOOH), in presence of solvent at 40° C. to obtain the compound of formula (II).

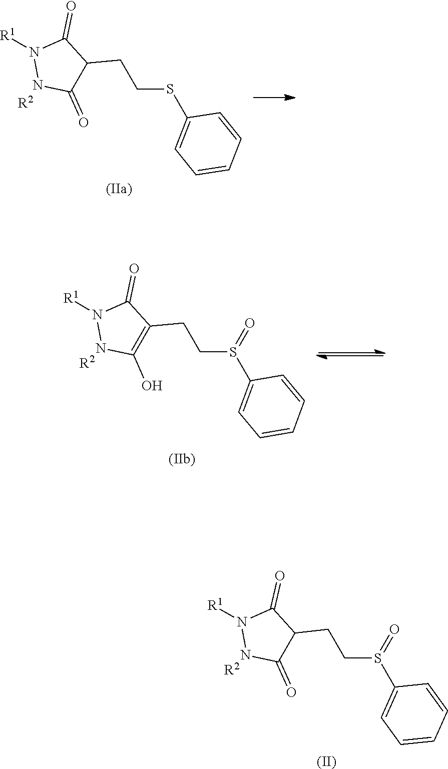

The solvent used in step (a) of preparation of compound of formula (II) is alcoholic solvent, THF, ACN or a mixture thereof.

The solvent used in step (b) of preparation of compound of formula (II) is ACN, THF, alcoholic solvent, or a mixture thereof.

The present invention relates to a process of preparing sulfinpyrazone drug comprising the steps of:
a) reacting the example 1 with thiophenol (PhSH) as a nucleophile in the presence of sodium methoxide solution (NaOMe), and solvent at room temperature for 2 hours to obtain a sulfide compound;

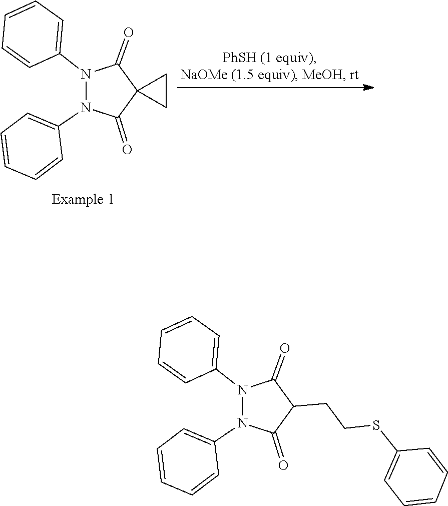 [SMILES: O=C1N(c2ccccc2)N(c2ccccc2)C(=O)C12CC2 ; O=C1C(CCSc2ccccc2)C(=O)N(c2ccccc2)N1c1ccccc1]

b) oxidising the sulfide compound with methanesulfonic acid (MsOH), and tert-butyl hydroperoxide (tBuOOH), in presence of solvent at 40° C. to obtain the sulfinpyrazone;

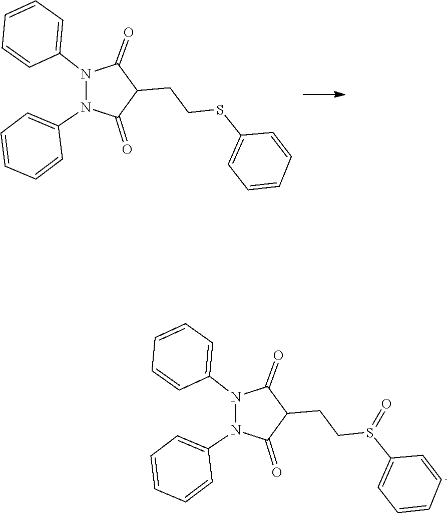 [SMILES: O=C1C(CCSc2ccccc2)C(=O)N(c2ccccc2)N1c1ccccc1 ; O=C1C(CCS(=O)c2ccccc2)C(=O)N(c2ccccc2)N1c1ccccc1]

The solvent used in step (a) is alcoholic solvent, THF, ACN or a mixture thereof.

The solvent used in step (b) is ACN, THF, alcoholic solvent, or a mixture thereof.

The cost is reduced upto 50% for preparing sulfinpyrazone.

It is submitted that the cyclopropyl ring of formula I compounds is crucial and important in terms of chemistry, and for subsequent reaction of preparation of sulfinpyrazone (and other drugs or APIs as mentioned herein), as said cyclopropyl ring get easily opened up by nucleophilic reaction. Such case is not favorable for other cyclic rings like cyclobutyl, cyclopentyl, etc. and for simple linear chains like methyl, ethyl, propyl, etc.

The present invention provides a process of preparation of sulfinpyrazone derivatives or analogues, comprising the steps of.
i. reacting the example 1 with thiophenol (PhSH) as a nucleophile in the presence of sodium methoxide solution (NaOMe), and solvent at room temperature for 2 hours to obtain a sulfide compound (formula IIa); (ii) oxidizing the sulfide compound with methanesulfonic acid (MsOH), and tert-butyl hydroperoxide (tBuOOH), in presence of solvent at 40° C. to obtain the sulfinpyrazone (formula II).

The solvent used in step (i) is alcoholic solvent, THF, ACN or a mixture thereof. In another aspect of the present invention, the solvent used in step (ii) is ACN, THF, alcoholic solvent, or a mixture thereof.

The present invention involves hypervalent iodine mediated metal-free intramolecular N—N bond formation of specific dianilides, together with its application to synthesize pharmaceutical molecule sulfinpyrazone without using highly toxic and expensive hydrazines,

Also, the reactivity pattern (while preparing the compounds of Formula I) of the substrate, and a complete inhibition of the reaction in the presence of 0.5-2.5 equiv. of BHT and TEMPO indicates the involvement of N-centred amidyl diradicals.

Specifically, the intramolecular dehydrogenative N—N bond formation is a key-step in the construction of the pyrazolidine-3,5-dione core of sulfinpyrazone class of drugs. In this regard, the present invention provides a highly efficient method for metal-free intramolecular dehydrogenative N—N bond formation of easily accessible dianilide precursors (i.e., formula I compound acting as dianilide precursor).

More specifically, the present invention provides an in situ preparation of diphenylhydrazine via N—N bond formation from inexpensive starting material aniline which leads to the pyrazolidine-3,5-dione core under very mild reaction condition followed by the synthesis of sulfinpyrazone class of drugs via smooth functionalization of well-designed diversity oriented cyclopropyl key intermediate.

### EXAMPLES

Following examples are given by way of illustration and therefore should not be construed to limit the scope of the invention.

### Materials

All reagents and solvents were used as received from commercial sources unless and other-wise noted. All experiments were carried out in a round bottom flask or Schlenk tube equipped with a stirring bar. Aluminium plates precoated with silica gel 60 PF254, 0.25 mm or 0.5 mm, were utilized for thin-layer chromatography (TLC) to monitor the progress of a reaction. Visualization of the developed TLC plate was performed by irradiation with UV light. Column chromatographic purifications were carried out on flash silica gel (240-400 mesh) using ethyl acetate, acetone, DCM and petroleum ether as eluents. The 1H and 13C NMR spectra were recorded on 400/500 MHz and 100/125 MHz NMR spectrometers respectively, in CDCl3 or DMSO-d6. Chemical shifts were reported as δ values from standard peaks. The multiplicities of signals are designated by the following abbreviations: s (singlet), d (doublet), t (triplet), q (quartet), quint. (quintet), m (multiplet). Coupling constants (J) are reported in hertz. Melting points are uncorrected. High-resolution mass spectrometry HRMS) was performed on a TOF/Q-TOF mass spectrometer. The substrate cyclopropane-1,1-dicarboxylic acid 7 was prepared using known literature procedure.1 The dianildies 5y and 5z were prepared as per the literature procedure.

### Example 1: General Experimental Procedure for the Synthesis of Dianilides (Compounds 5a-s, 5w, 5x)

An oven dried two-neck round bottom flask was charged with cyclopropane-1,1 dicarboxylic acid 7 (1.54 mmol, 1 equiv.) and thionyl chloride (5 ml) under argon. After overnight stirring at refluxing condition (90° C.), the excess of thionyl chloride was removed by distillation, yielding the dichloride 7′ as a yellow oil. The product was used in the next step without further purification. To the solution of cyclopropane-1,1-dicarbonylchloride 7′ (1.54 mmol, 1 equiv.) in THF (10 ml), the solution of amines 6a-u (3.85 mmol, 2.5 equiv.) and triethyl amine (4.62 mmol, 3 equiv.) in THF (5 ml) was added dropwise at 0° C. temperature with vigorous stirring. Combination of these two solutions caused the precipitation of triethylamine hydrochloride as a finely dispersed powder. After two hours stirring at room temperature, the reaction mixture was diluted with water (15 mL) and extracted with EtOAc (3×30 mL). The organic layer was separated and washed with brine solution once and dried over anhydrous Na2SO4. Evaporation of the solvent under vacuo to dryness followed by the purification of the crude product using column chromatography pet ether:ethyl acetate (4:1 to 1:4) provided the expected dianilides 5a-s, 5w, 5x in very good yields.

The known dianilides 5a-d, 5g, 5i-k, 5m, 5o, 5q-r, 5w were prepared by the same procedure as provided above and their structure was confirmed by comparing their analytical data with the reported literature (refer, (a) L.-J. Min, Z.-H. Shen, J. Bajsa-Hirschel, C. L. Cantrell, L. Han, X.-W. Hua, X.-H. Liu and S. O. Duke, PESTIC BIOCHEM PHYS, 2022, 188, 105228; (b) X. Liu, Y. Wen, Z. Shen, J. Weng and C. Tan, Faming Zhuanli Shenqing, 2020, CN 112142619 A; and (c) V. Karaluka, R. M. Lanigan, P. M. Murray, M. Badlandc and T. D. Org. Biomol. Chem., 2015, 13, 10888.

### Synthesis of Dianilides 5t-u

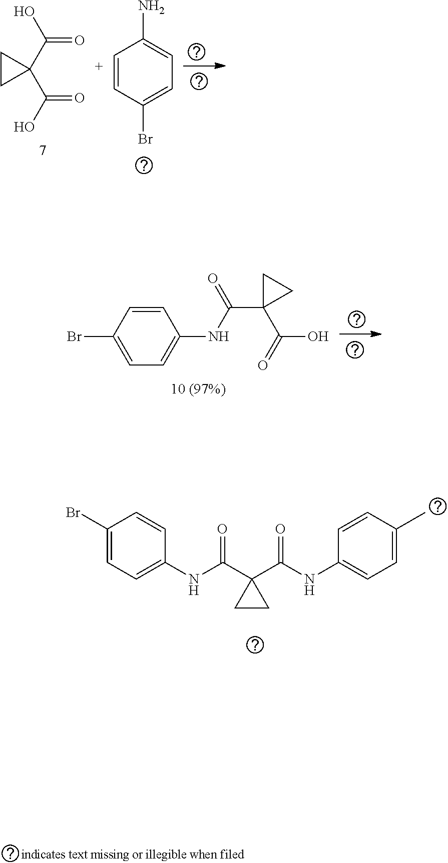

The intermediate 10 was prepared by following the reported procedure and used for the next step directly. Similarly, dianilides 5t and 5u were synthesized following the reported procedure by slightly modifying the coupling reagent (refer, Z. Zhan, J. Ai, Q. Liu, Y. Ji, T. Chen, Y. Xu, M. Geng, and W. Duan, ACS Med. Chem. Lett., 2014, 5, 673).

### Synthesis of Dianilides 5v

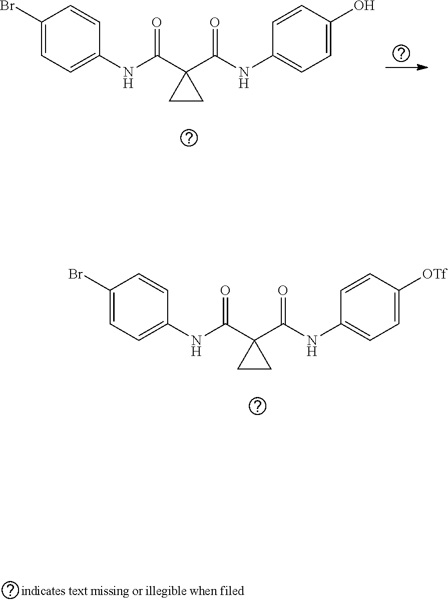 [SMILES: O=C(Nc1ccc(O)cc1)C1(C(=O)Nc2ccc(Br)cc2)CC1 ; O=C(Nc1ccc(Br)cc1)C1(C(=O)Nc2ccc(OS(=O)(=O)C(F)(F)F)cc2)CC1]

Dianilide 5v was synthesized by treatment of triflic anhydride with the danilide 5u using known literature procedure (refer, 5. T. Gieshoff, A. Kehl, D. Schollmeyer, K. D. Moeller and S. R. Waldvogel, J. Am. Chem. Soc., 2017, 139, 12317).

### Example 2: General Experimental Procedure for the Preparation of Pyrazolidine-3,5-Dione Derivatives

To an oven dried Schlenk tube, dianilide (50 mg, 1 equivalent) and diacetoxyiodobenzene (2 equivalent) were added under the gentle stream of argon and flushed with argon gas followed by the addition of dry acetonitrile (0.1 M). The reaction mixture was placed on preheated oil bath at 70° C. and stirred for 16 hours. After completion of the reaction (TLC), the reaction was cooled to room temperature and evaporated the solvent a rotatory evaporator and the residue was purified by flash silica gel column chromatography using a gradient of ethyl acetate: pet ether to afford the corresponding pyrazolidine-3,5-dione derivatives in good to excellent yield.

### Example 3: Experimental Procedure for the Synthesis of Dianilide 11

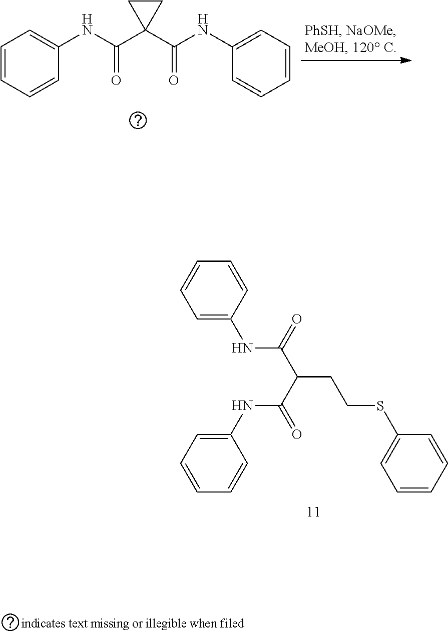 [SMILES: O=C(Nc1ccccc1)C1(C(=O)Nc2ccccc2)CC1 ; O=C(Nc1ccccc1)C(CCSc1ccccc1)C(=O)Nc1ccccc1]

An oven dried pressure tube was charged with sodium methoxide (5.8 mg, 0.11 mmol, 1.5 equiv.) under argon atmosphere. Dry methanol (0.7 ml, 0.1 M) followed by the thiophenol (7.9 mg, 0.07 mmol, 1 equiv.) was added and the reaction mixture was kept for 30 min. at room temperature before adding the dianilide 5a (20 mg, 0.07 mmol, 1 equiv.). After stirring the reaction mixture at 120° C. for the completion of the reaction (monitored by TLC, approx. 12h), the solvent was evaporated and the residue was mixed with water (5 ml) and EtOAc (5 ml). The aqueous part was extracted with EtOAc (3×5 ml) and the combined organic layers were dried over Na2SO4 and concentrated under reduced pressure. The resulting crude mixture was purified by flash column chromatography using pet ether:ethyl acetate (4:1) to provide pure sulfide compound 11 in 81% (16.6 mg) yield as a colorless sticky solid.

TABLE 1
Optimization of Reaction Conditions to obtain 3aª

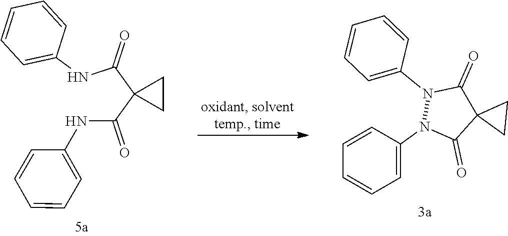

| Sr. | | | Temp. | Time | Yield^{b} |
|---|---|---|---|---|---|
| no | Conditions | Solvent | (°C.) | (h) | (%) |
| 1. | KMnO₄ (2.5 equiv.) | acetone | 60 | 24 | NR |
| 2. | CuBr₂ (20 mol %), O₂ | DMSO | 120 | 24 | NR |
| 3. | [Mes-acr]⁺ BF₄⁻, ( 1 mol %) | HFIP | 25 | 24 | NR |
| 4. | PIDA (1 equiv.) | HFIP | 40 | 16 | 30 |
| | | | | | (43)^{c} |
| 5. | PIDA (1 equiv.) | HFIP | 70 | 16 | 24 |
| 6. | PIDA (1.5 equiv.) | HFIP | 70 | 16 | 30 |
| 7. | PIDA (2 equiv.) | HFIP | 70 | 16 | 40 |
| 8. | PhIO (2 equiv.) | HFIP | 70 | 16 | 38 |
| 9. | PIFA (2 equiv.) | HFIP | 70 | 16 | 15 |
| 10. | PIDA (2 equiv.) | DMF/EtOH/ | 70 | 16 | NR |
| | | THF/IPA/ | | | |
| | | MeOH/DCE/^{t}BuOH | | | |
| 11. | PIDA (2 equiv.) | ACN | 70 | 16 | 56 |
| | | | | | (74)^{c} |
| 12. | PhIO (2 equiv.) | ACN | 70 | 16 | 41 |
| | | | | | (64)^{c} |
| 13. | PhI (20 mol %), m-CPBA (1.2 | ACN | 25 | 12 | 44 |
| | equiv.) | | | | |
| 14. | PhI (20 mol %), Oxone (3 | HFIP | 70 | 17 | NR |
| | equiv.) | | | | |
| 15. | PIDA (2 equiv.), HFIP (3 | MeOH | 70 | 16 | 24 |
| | equiv.) | | | | |
| 16. | PIDA (2 equiv.), HFIP (3 | toluene | 70 | 16 | 34 |
| | equiv.) | | | | |
| 17. | PIDA (2 equiv.), HFIP (3 | ACN | 70 | 16 | 68 |
| | equiv.) | | | | (72)^{c} |
| 18. | PIDA (2 equiv.) | HFIP:MeOH | 70 | 16 | 37 |
| | | (1:1) | | | |
| 19. | PIDA (2 equiv.) | HFIP:toluene | 70 | 16 | 20 |
| | | (1:1) | | | |
| 20. | PIDA (2equiv.) | HFIP:heptane | 70 | 16 | 38 |
| | | (1:1) | | | |
| 21. | PIDA (2 equiv.) | HFIP:hexane (1:1) | 70 | 16 | 30 |
| 22. | PIDA (2 equiv.) | HFIP:ACN (1:1) | 70 | 16 | 61(75)^{c} |
| 23. | PIDA (2 equiv.), under argon | Dry ACN | 70 | 16 | 87 |
| | | | | | (92)^{c} |
| 24 | PIDA (2 equiv.), under argon, | ACN | 70 | 16 | 61 |
| | 4AºMS | | | | (74)^{c} |
| 25 | PIDA (2 equiv.), under argon, | ACN | 70 | 16 | 63 |
| | 3AºMS | | | | (71)^{c} |
| 26 | PhIO (2 equiv.), under argon | Dry ACN | 70 | 16 | 23 |
| 27. | IBX (2 equiv.), under argon | Dry ACN | 70 | 16 | NR |
| 28. | DMP (2 equiv.), under argon | Dry ACN | 70 | 16 | NR |
| 29. | PhI (20 mol %), m-CPBA (3 | Dry ACN | 70 | 16 | 26 |
| | equiv) | | | | |
| 30. | PhI (20 mol %), Oxone (3 | Dry ACN | 70 | 16 | NR |
| | equiv) | | | | |

^{a}Reaction conditions: 5a (20 mg, 1.0 equiv.), Oxidant in solvent (0.1M, 0.7 ml).
^{b}Isolated yield.
^{c}Yield in the parentheses is based on the recovered starting material.

### Example 4: General Experimental Procedure for the Preparation of Pyrazolidine-3,5-Dione Derivatives (Compounds 3a-z)

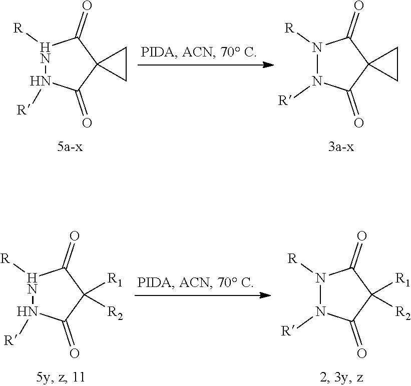

To an oven dried Schlenk tube containing dianilide 5a-z, 11 (50 mg, 1 equiv.) and diacetoxyiodobenzene (2 equiv.) under argon was added dry acetonitrile (0.1 M). The reaction mixture was placed in a preheated oil bath at 70° C. and stirred for 16 hours. After completion of the reaction (TLC) it was cooled to room temperature and the solvent was evaporated on a rotatory evaporator. The residue was purified by flash silica gel column chromatography using a gradient of pet ether:ethyl acetate (4:1 to 3:2) to afford the corresponding pyrazolidine-3,5-dione derivatives 2, 3a-z in good to excellent yield.

### Example 5: Typical Experimental Procedure for the Preparation of Representative Product 3a

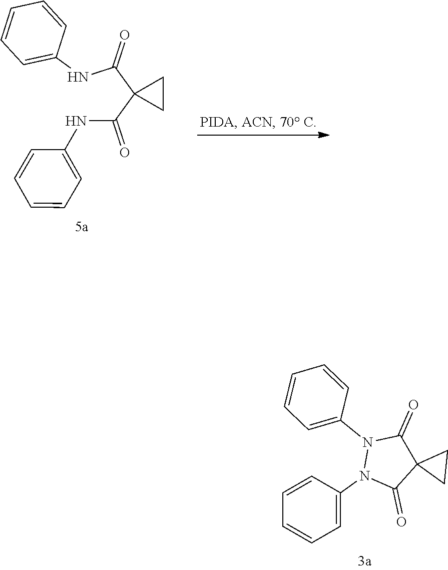 [SMILES: O=C(Nc1ccccc1)C1(C(=O)Nc2ccccc2)CC1 ; O=C1N(c2ccccc2)N(c2ccccc2)C(=O)C12CC2]

To an oven dried Schlenk tube containing dianilide 5a (50 mg, 0.18 mmol, 1 equiv.) and diacetoxyiodobenzene (115 mg, 0.36 mmol, 2 equiv.) was added dry acetonitrile (1.8 ml, 0.1 M). The reaction mixture was placed on preheated oil bath at 70° C. and stirred for 16 hours. After completion of the reaction (TLC) it was cooled to room temperature and the solvent was evaporated on a rotatory evaporator. The residue was purified by flash silica gel column chromatography using a gradient of pet ether:ethyl acetate (6:1) to afford the corresponding pyrazolidine-3,5-dione derivative 3a as a white solid in 87% yield (43.2 mg) and in based on the recovery of starting material 92% yield.

### Example 6: Gram Scale Experimental Procedure for the Preparation of Representative Product 3a

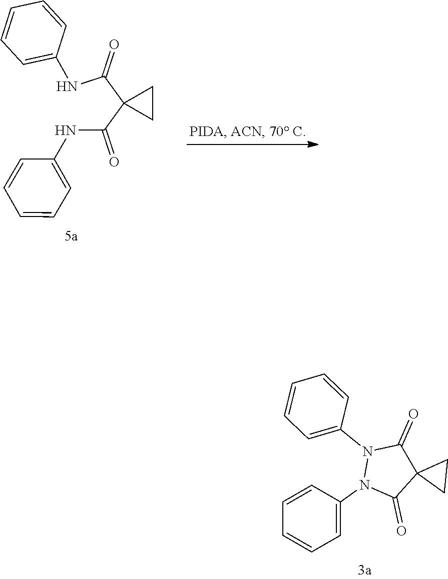 [SMILES: O=C(Nc1ccccc1)C1(C(=O)Nc2ccccc2)CC1 ; O=C1N(c2ccccc2)N(c2ccccc2)C(=O)C12CC2]

To an oven dried Schlenk tube containing dianilide 5a (1 gm, 3.6 mmol, 1 equiv.) and diacetoxyiodobenzene (2.3 g, 7.14 mmol, 2 equiv.) was added dry acetonitrile (36 ml, 0.1 M). The reaction mixture was placed on preheated oil bath at 70° C. and stirred for 24 hours. After completion of the reaction (TLC) it was cooled to room temperature and the solvent was evaporated on a rotatory evaporator. The residue was purified by flash silica gel column chromatography using a gradient of pet ether:ethyl acetate (6:1) to afford the corresponding pyrazolidine-3,5-dione derivative 3a as a white solid in 63% yield (0.626 g) and in based on the recovery of starting material 67% yield.

### Example 7: Preparation of Intermediate Compound (I-1)

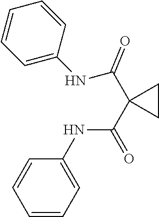 [SMILES: O=C(Nc1ccccc1)C1(C(=O)Nc2ccccc2)CC1]

An oven dried two-neck round bottom flask was charged with cyclopropane-1,1-dicarboxylic acid (200 mg, 1.54 mmol). The flask was flushed twice with argon gas and mixed with thionyl chloride (5 ml) under argon. The reaction was stirred at reflux (90° C.) for overnight. The excess of thionyl chloride was removed by distillation, yielding the dichloride as yellow oil. The product was used in the next step of the reaction without further purification. To the solution of cyclopropane-1,1-dicarbonylchloride (1.54 mmol) in THF (10 ml), aniline (3.85 mmol, 2.5 equivalent) and triethylamine (4.62 mmol, 3 equivalent) in THF (5 ml) was added dropwise at 0° C. temperature with vigorous stirring. Combination of these two solutions caused the precipitation of triethylammonium chloride as a finely dispersed powder. After two hours stirring at room temperature, the reaction mixture was diluted with water (15 mL) and extracted with EtOAc (3×30 mL) three times. The organic layer was separated and washed with brine solution once and dried over anhydrous Na2SO4. Evaporation of the solvent under vacuo to dryness followed by the purification of the crude product using column chromatography (pet ether:ethyl acetate, 4:1) provided the expected dianilide in very good yield.

¹H NMR (400 MHz, CDCl₃) δ 10.03 (s, 1H), 7.60 (d, J=7.9 Hz, 4H), 7.30 (t, J=7.8 Hz, 4H), 7.07 (t, J=7.3 Hz, 2H), 1.48 (s, 4H); HRMS (ESI-TOF) m/z: [M+H]⁺ calcd for C₁₇H₁₇O₂N₂ 281.1285, found 228.1281.

The following intermediate compounds are prepared by following the above experimental procedure with minor non-critical variations.

| Intermediate | | |
|---|---|---|
| Compounds | Structure | Characterization data |
| I-2 | 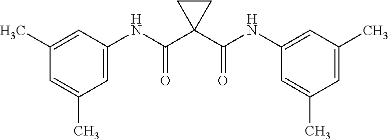 | ¹H NMR (400 MHz, CDCl₃) δ 8.87 (brs, 2H), 7.16 (s, 4H), 6.79 (s, 2H), 2.31 (s, 12H), 1.61 (s, 4H); ¹³C NMR (100 MHz, CDCl₃) δ 168.7, 138.7, 137.1, 126.5, 118.4, 29.6, 21.3, 17.0. |
| I-3 | 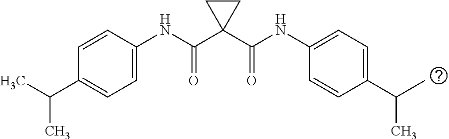 | ¹H NMR (400 MHz, CDCl₃) δ 8.96 (brs, 2H), 7.42 (d, J = 8.5 Hz, 4H), 7.20 (d, J = 8.4 Hz, 4H), 2.89 (septet, J = 6.9 Hz, 2H), 1.61 (s, 4H), 1.25 (s, 6H), 1.23 (s, 6H); ¹³C NMR (100 MHz, CDCl₃) δ 168.8, 145.6, 134.9, 126.9, 120.8, 33.6, 29.6, 24.0, 17.0; HRMS (ESI-TOF) m/z: [M + H]⁺ calcd for C₂₃H₂₉O₂N₂ |
| | | 365.2224, found 365.2224. |
| I-3 | 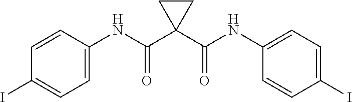 [SMILES: O=C(Nc1ccc(I)cc1)C1(C(=O)Nc2ccc(I)cc2)CC1] | ¹H NMR (400 MHz, DMSO-d⁶) δ 10.09 (brs, 2H), 7.63 (d, J = 8.8 Hz, 4H), 7.46 (d, J = 8.7 Hz, 4H), 1.43 (s, 4H); ¹³C NMR (100 MHz, DMSO-d⁶) δ 168.1, 138.8, 137.1, 122.5, 87.2, 32.1, 15.4; HRMS (ESI-TOF) m/z: [M + H]⁺ calcd for |
| | | C₁₇H₁₅O₂N₂I₂S 532.9217, found |
| | | 532.9210. |
| I-4 | 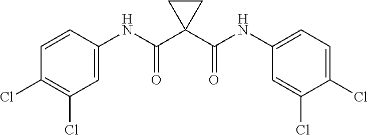 [SMILES: O=C(Nc1ccc(Cl)c(Cl)c1)C1(C(=O)Nc2ccc(Cl)c(Cl)c2)CC1] | ¹H NMR (400 MHz, DMSO-d⁶) δ 10.28 (brs, 2H), 8.11-7.95 (m, 2H), 7.56-7.54 (m, 4H), 1.44 (s, 4H); ¹³C NMR (100 MHz, DMSO-d⁶) δ 168.0, 139.2, 130.7, 130.4, 124.9, 121.5, 120.2, 32.4, 15.4; HRMS (ESI-TOF) m/z: [M + H]⁺calcd for C₁₇H₁₃O₂N₂Cl₄ 416.9726, found 416.9723. |
| I-5 | 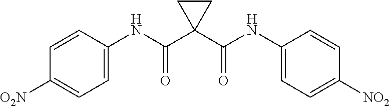 [SMILES: O=C(Nc1ccc([N+](=O)[O-])cc1)C1(C(=O)Nc2ccc([N+](=O)[O-])cc2)CC1] | ¹H NMR (400 MHz, DMSO-d⁶) δ 10.61 (brs, 2H), 8.24-8.18 (m, 4H), 7.91-7.86 (m, 4H), 1.51 (s, 4H); ¹³C NMR (100 MHz, DMSO-d⁶) δ 168.5, 145.4, 142.5, 124.8, 119.9, 33.1, 15.8. |
| I-6 | 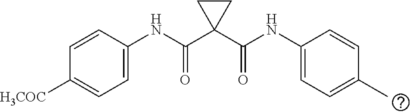 | ¹H NMR (400 MHz, DMSO-d⁶) δ 10.32 (brs, 2H), 7.92 (d, J = 8.8 Hz, 4H), 7.78 (d, J = 8.8 Hz, 4H), 2.53 (s, 6H), 1.50 (s, 4H); ¹³C NMR (100 MHz, DMSO-d⁶) δ 196.6, 168.3, 26.4, 15.6; HRMS (ESI-TOF) m/z: [M + H] calcd for C₂₁H₂₁O₄N₂ |
| | | 365.1496, found 365.1494. |
| I-7 | 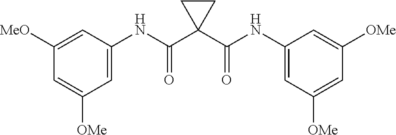 [SMILES: COc1cc(NC(=O)C2(C(=O)Nc3cc(OC)cc(OC)c3)CC2)cc(OC)c1] | ¹H NMR (400 MHz, DMSO-d⁶) δ 9.90 (brs, 2H), 6.91 (d, J = 2.1 Hz, 4H), 6.22 (t, J = 2.3 Hz, 2H), 3.70 (s, 12H), 1.43 (s, 4H); ¹³C NMR (100 MHz, DMSO-d⁶) δ 168.2, 160.3, 140.6, 98.6, 95.8, 55.1, 32.0, 15.3; HRMS (ESI-TOF) m/z: [M + H]⁺ calcd for C₂₁H₂₅O₆N₂ 401.1707, found 401.1704. |
| I-8 | 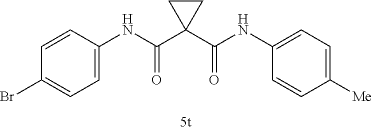 [SMILES: Cc1ccc(NC(=O)C2(C(=O)Nc3ccc(Br)cc3)CC2)cc1] | ¹H NMR (400 MHz, DMSO-d⁶) δ 10.17 (brs, 1H), 9.88 (brs, 1H), 7.60 (d, J = 7.9 Hz, 2H), 7.46 (d. J = 7.9 Hz, 4H), 7.10 (d, J = 7.9 Hz, 2H), 2.25 (s, 3H), 1.45 (s, 4H); ¹³C NMR (100 MHz, DMSO-d⁶) δ 168.4, 167.9, 138.2, 136.2, 132.6, 131.3, 128.8, 122.3, 120.6, 115.2, 31.5, |
| | | 20.4, 15.5; HRMS (ESI-TOF) m/z: |
| | | [M + H]⁺calcd for C₁₈H₁₈BrN₂O₂ |
| | | 373.0546, found 373.0553. |
| I-9 | 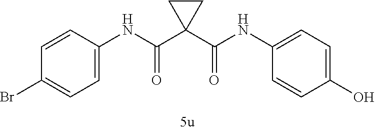 [SMILES: O=C(Nc1ccc(O)cc1)C1(C(=O)Nc2ccc(Br)cc2)CC1] | ¹H NMR (400 MHz, DMSO-d⁶) δ 10.27 (brs, 1H), 9.66 (brs, 1H), 9.21 (brs, 1H), 7.60 (d, J = 8.0 Hz, 2H), 7.46 (d, J = 8.1 Hz, 2H), 7.34 (d, J = 7.9 Hz, 2H), 6.68 (d, J = 8.0 Hz, 2H), 1.44 (s, 4H); ¹³C NMR (100 MHz, DMSO-d⁶) δ 168.4, 167.8, 153.8, 138.2, 131.3, 130.1, 122.6, |
| | | 122.2, 115.1, 114.8, 31.1, 15.5; |
| | | HRMS (ESI-TOF) m/z: |
| | | [M + H]⁺ calcd for C₁₇H₁₆BrN₂O₃ |
| | | 375.0339, found 375.0333. |
| I-10 | 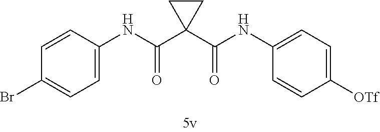 [SMILES: O=C(Nc1ccc(Br)cc1)C1(C(=O)Nc2ccc(OS(=O)(=O)C(F)(F)F)cc2)CC1] | ¹H NMR (400 MHz, DMSO-d⁶) δ 10.28, (brs, 1H), 10.10 (brs, 1H), 7.79 (d, J = 8.5 Hz, 2H), 7.60 (d, J = 8.0 Hz, 2H), 7.56-7.36 (m, 4H), 1.45 (s, 4H); ¹³C NMR (100 MHz, DMSO-d⁶) δ 168.2, 167.9, 144.4, 139.4, 138.3, 131.3, 122.3, 121.8, 121.6, 118.3 (q, J = 320.4 Hz, CF₃), 115.2, 32.0, 15.4; HRMS (ESI- |
| | | TOF) m/z: [M + H]⁺ calcd for |
| | | C₁₈H₁₅BrF₃N₂O₅S 506.9832, found |
| | | 506.9832. |
| I-11 | 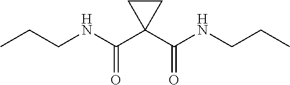 | ¹H NMR (400 MHz, CDCl₃) δ 7.11 (brs, 2H), 3.21 (q, J = 6.1 Hz, 4H), 1.53 (sextet, J = 7.1 Hz, 4H), 1.35 (s, 4H), 0.92 (t, J = 7.4 Hz, 6H); ¹³C NMR (100 MHz, CDCl₃) δ 170.7, 41.5, 28.2, 22.6, 16.1, 11.4; HRMS |
| | | (ESI-TOF) m/z: [M + H]⁺ calcd for |
| | | C₁₁H₂₁O₂N₂ 213.1598, found |
| | | 213.1600. |
| I-12 | 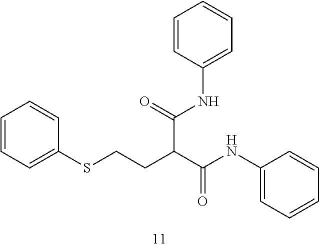 [SMILES: O=C(Nc1ccccc1)C(CCSc1ccccc1)C(=O)Nc1ccccc1] | ¹H NMR (200 MHz, CDCl₃) δ 9.07 (brs, 2H), 7.56 (d, J = 7.6 Hz, 4H), 7.36-7.31 (m, 6H), 7.23-7.13 (m, 5H), 3.74 (t, J = 7.5 Hz, 1H), 3.06 (t, J = 7.0 Hz, 2H), 2.39 (q, J = 7.1 Hz, 2H); ¹³C NMR (100 MHz, CDCl₃) δ 168.7, 137.2, 134.8, 129.9, 129.05, 129.01, 126.6, 124.9, 120.3, 54.7, 32.5, 31.7; HRMS (ESI-TOF) m/z: [M + H]⁺ calcd for C₂₃H₂₃N₂O₂S 391.1475, found 391.1475. |

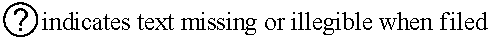

### Example 8: Preparation of 5,6-Diphenyl-5,6-diazaspiro[2.4]heptane-4,7-dione

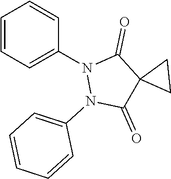 [SMILES: O=C1N(c2ccccc2)N(c2ccccc2)C(=O)C12CC2]

To an oven dried Schlenk tube, the intermediate compound 1-1 (1 gm, 3.6 mmol, 1 equivalent) and diacetoxyiodobenzene (2.3 g, 7.14 mmol, 2 equivalent) were added under the gentle stream of argon and flushed with argon gas followed by the addition of dry acetonitrile (36 ml, 0.1 M). The reaction mixture was placed on preheated oil bath at 70° C. and stirred for 24 hours. After completion of the reaction (TLC), the reaction was cooled to room temperature and evaporated the solvent a rotatory evaporator and the residue was purified by flash silica gel column chromatography using a gradient of ethyl acetate-petroleum ether to afford the title compound.

¹H NMR (400 MHz, CDCl₃) δ 7.43-7.30 (i, 8H), 7.23-7.15 (M, 2H), 1.92 (s, 4H); ¹³C NMR (100 MHz, CDCl₃) δ 171.2, 136.4, 128.9, 126.5, 122.2, 26.9, 21.8; HRMS (ESI-TOF) m/z: [M+H]-calcd for C₁₇H₁₅O₂N₂ 279.1128, found 279.1126.

The following examples were prepared by following the experimental procedure of example-1 with appropriate starting materials and minor non-critical changes.

| Examples | Structure | Characterization data |
|---|---|---|
| 2 | 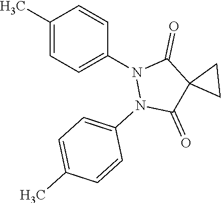 [SMILES: Cc1ccc(N2C(=O)C3(CC3)C(=O)N2c2ccc(C)cc2)cc1] | ¹H NMR (400 MHz, CDCl₃) δ 7.24 (d, J = 8.4 Hz, 4H), 7.13 (d, J = 8.4 Hz, 4H), 2.29 (s, 6H), 1.89 (s, 4H); ¹³C NMR (100 MHz, CDCl₃) δ 171.2, 136.5, 133.8, 129.5, 122.6, 26.8, 21.5, 21.0; HRMS (ESI- TOF) m/z: [M + H]⁺ calcd for C₁₉H₁₉O₂N₂ 307.1441, found 307.1435. |
| 3 | 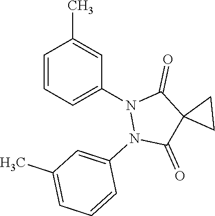 [SMILES: Cc1cccc(N2C(=O)C3(CC3)C(=O)N2c2cccc(C)c2)c1] | ¹H NMR (400 MHz, CDCl₃) δ 7.24 (s, 2H), 7.20 (t, J = 7.8 Hz, 2H), 7.12 (d, J = 8.3 Hz, 2H), 6.99 (d, J = 7.5 Hz, 2H), 2.33 (s, 6H), 1.90 (s, 4H); ¹³C NMR (100 MHz, CDCl₃) δ 171.3, 138.9, 136.4, 128.7, 127.4, 123.3, 119.4, 26.9, 21.7, 21.4; HRMS (ESI-TOF) m/z: [M + H]⁺ calcd for C₁₉H₁₉O₂N₂ 307.1441, found 307.1438. |
| 4 | 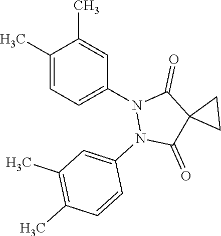 [SMILES: Cc1ccc(N2C(=O)C3(CC3)C(=O)N2c2ccc(C)c(C)c2)cc1C] | ¹H NMR (400 MHz, CDCl₃) δ 7.20 (d, J = 1.3 Hz, 2H), 7.08-7.01(m, 4H), 2.22 (s, 6H), 2.18 (s, 6H), 1.87 (s, 4H); ¹³C NMR (100 MHz, CDCl₃) δ 171.4, 137.3, 135.4, 134.1, 129.9, 124.3, 120.2, 26.8, 21.4, 19.9, 19.3; HRMS (ESI-TOF) m/z: [M + H]⁺ calcd for C₂₁H₂₃O₂N₂ 335.1754, found 335.1756. |
| 5 | 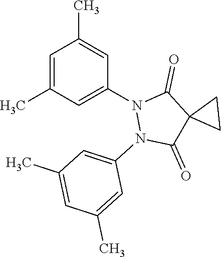 [SMILES: Cc1cc(C)cc(N2C(=O)C3(CC3)C(=O)N2c2cc(C)cc(C)c2)c1] | ¹H NMR (400 MHz, CDCl₃) δ 6.99 (s, 4H), 6.82 (s, 2H), 2.27 (s, 12H), 1.87 (s, 4H); ¹³C NMR (100 MHz, CDCl₃) δ 171.5, 138.6, 136.4, 128.6, 120.5, 26.8, 21.5, 21.3; HRMS (ESI-TOF) m/z: [M + H]⁺ calcd for C₂₁H₂₃O₂N₂ 335.1754, found 335.1755. |
| 6 | 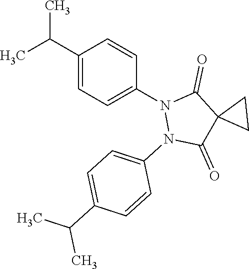 [SMILES: CC(C)c1ccc(N2C(=O)C3(CC3)C(=O)N2c2ccc(C(C)C)cc2)cc1] | ¹H NMR (400 MHz, CDCl₃) δ 7.29 (d, J = 8.5 Hz, 4H), 7.18 (d, J = 8.4 Hz, 4H), 2.86 (septate, J = 6.9 Hz, 2H), 1.89 (s, 4H), 1.21 (s, 6H), 1.19 (s, 6H); ¹³C NMR (100 MHz, CDCl₃) δ 171.4, 147.2, 134.1, 126.9, 122.3, 33.6, 26.9, 23.8, 21.7; HRMS (ESI- TOF) m/z: [M + H]⁺ calcd for C₂₃H₂₇O₂N₂ 363.2067, found 363.2069. |
| 7 | 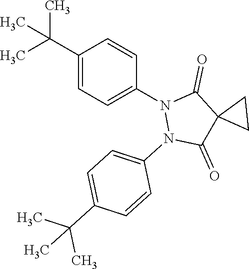 [SMILES: CC(C)(C)c1ccc(N2C(=O)C3(CC3)C(=O)N2c2ccc(C(C)(C)C)cc2)cc1] | ¹H NMR (500 MHz, CDCl₃) δ 7.35 (d, J = 8.2 Hz, 4H), 7.30 (d, J = 8.5 Hz, 4H), 1.89 (s, 4H), 1.27 (s, 18H); ¹³C NMR (125 MHz, CDCl₃) δ 171.5, 149.4, 133.9, 125.8, 121.8, 34.5, 31.2, 26.8, 21.7; HRMS (ESI-TOF) m/z: [M + H]⁺ calcd for C₂₅H₃₁O₂N₂ 391.2380, found 391.2385. |
| 8 | 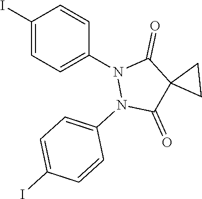 [SMILES: O=C1N(c2ccc(I)cc2)N(c2ccc(I)cc2)C(=O)C12CC2] | ¹H NMR (400 MHz, CDCl₃) δ 7.66 (d, J = 8.9 Hz, 4H), 7.10 (d, J = 8.9 Hz, 4H), 1.94 (s, 4H); ¹³C NMR (100 MHz, CDCl₃) δ 171.0, 138.1, 136.0, 123.6, 91.1, 26.8, 22.4; HRMS (ESI-TOF) m/z: [M + H]⁺ calcd for C₁₇H₁₃O₂N₂I₂ 530.9061, found 530.9064. |
| 9 | 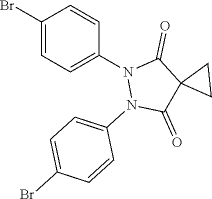 [SMILES: O=C1N(c2ccc(Br)cc2)N(c2ccc(Br)cc2)C(=O)C12CC2] | ¹H NMR (400 MHz, CDCl₃) δ 7.52-7.42 (m, 4H), 7.26-7.21 (m, 4H), 1.94 (s, 4H); ¹³C NMR (100 MHz, CDCl₃) δ 171.0, 135.3, 132.2, 123.5, 120.0, 26.8, 22.4; HRMS (ESI-TOF) m/z: [M + H]⁺ calcd for C₁₇H₁₃O₂N₂Br₂ 434.9338, found 434.9333. |
| 10 | 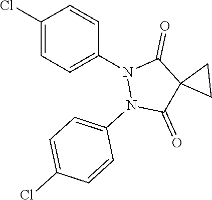 [SMILES: O=C1N(c2ccc(Cl)cc2)N(c2ccc(Cl)cc2)C(=O)C12CC2] | ¹H NMR (400 MHz, CDCl₃) δ 7.46-7.14 (m, 8H), 1.94 (s, 4H); ¹³C NMR (100 MHz, CDCl₃) δ 171.1, 134.7, 132.2, 129.2, 123.2, 26.8, 22.3; HRMS (ESI- TOF) m/z: [M + H]⁺ calcd for C₁₇H₁₃O₂N₂Cl₂ 347.0349, found 347.0346. |
| 11 | 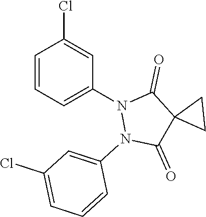 [SMILES: O=C1N(c2cccc(Cl)c2)N(c2cccc(Cl)c2)C(=O)C12CC2] | ¹H NMR (400 MHz, CDCl₃) δ 7.44 (t, J = 1.9 Hz, 2H), 7.32-7.26 (m, 2H), 7.25-7.17 (m, 4H), 1.96 (s, 4H); ¹³C NMR (100 MHz, CDCl₃) δ 171.2, 137.5, 134.9, 130.1, 126.9, 122.1, 119.8, 26.8, 22.5; HRMS (ESI-TOF) m/z: [M + H]⁺ calcd for C₁₇H₁₃O₂N₂Cl₂ 347.0349, found 347.0348. |
| 12 | 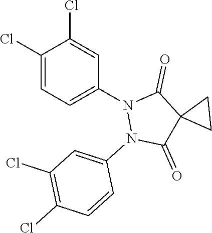 [SMILES: O=C1N(c2ccc(Cl)c(Cl)c2)N(c2ccc(Cl)c(Cl)c2)C(=O)C12CC2] | ¹H NMR (400 MHz, CDCl₃) δ 7.54 (d, J = 2.5 Hz, 2H), 7.43 (d, J = 8.6 Hz, 2H), 7.18 (dd, J = 8.7 & 2.5 Hz, 2H), 1.98 (s, 4H); ¹³C NMR (100 MHz, CDCl₃) δ 171.1, 135.5, 133.3, 130.8, 130.7, 123.6, 120.7, 26.7, 22.9; HRMS (ESI-TOF) m/z: [M + H]⁺ calcd for C₁₇H₁₁O₂N₂Cl₄ 414.9569, found 414.9562. |
| 13 | 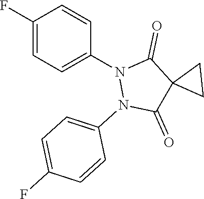 [SMILES: O=C1N(c2ccc(F)cc2)N(c2ccc(F)cc2)C(=O)C12CC2] | ¹H NMR (400 MHz, CDCl₃) δ 7.38-7.29 (m, 4H), 7.09-7.00 (m, 4H), 1.93 (s, 4H); ¹³C NMR (100 MHz, CDCl₃) δ 171.3, 160.9 (d, J = 247.2 Hz), 132.1 (d, J = 3.1 Hz), 124.4 (d, J = 8.4 Hz), 116.0 (d, J = 22.9 Hz), 26.7, 22.0; HRMS (ESI-TOF) m/z: [M + H]⁺ calcd for C₁₇H₁₃O₂N₂F₂ 315.0940, found 315.0939. |
| 14 | 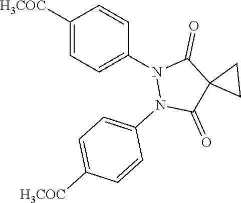 | ¹H NMR (400 MHz, CDCl₃) δ 8.03-7.86 (m, 4H), 7.56-7.35 (m, 4H), 2.56 (s, 6H), 2.0 (s, 4H); ¹³C NMR (100 MHz, CDCl₃) δ 196.6, 170.9, 140.2, 134.8, 129.4, 121.1, 27.0, 26.5, 22.8; HRMS (ESI-TOF) m/z: [M + H]⁺ calcd for C₂₁H₁₉O₄N₂ 363.1339, found 363.1341. |
| 15 | 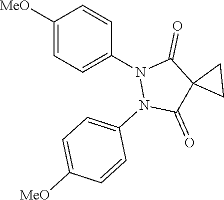 [SMILES: COc1ccc(N2C(=O)C3(CC3)C(=O)N2c2ccc(OC)cc2)cc1] | ¹H NMR (400 MHz, CDCl₃) δ 7.25 (d, J = 9.0 Hz, 4H), 6.84 (d, J = 9.0 Hz, 4H), 3.76 (s, 6H), 1.89 (s, 4H); ¹³C NMR (100 MHz, CDCl₃) δ 171.2, 158.3, 128.9, 125.2, 114.2, 55.4, 26.8, 21.3; HRMS (ESI- TOF) m/z: [M + H]⁺ calcd for C₁₉H₁₉O₄N₂ 339.1339, found 339.1344. |
| 16 | 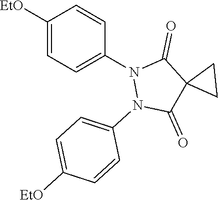 [SMILES: CCOc1ccc(N2C(=O)C3(CC3)C(=O)N2c2ccc(OCC)cc2)cc1] | ¹H NMR (400 MHz, CDCl₃) δ 7.26-7.20 (m, 4H), 6.85-6.79 (m, 4H), 3.97 (q, J = 7.0 Hz, 4H), 1.88 (s, 4H), 1.38 (t, J = 7.0 Hz, 6H); ¹³C NMR (100 MHz, CDCl₃) δ 171.2, 157.8, 128.7, 125.3, 114.7, 63.6, 26.8, 21.2, 14.7; HRMS (ESI-TOF) m/z: [M + H]⁺ calcd for C₂₁H₂₃O₄N₂ 367.1652, found 367.1647. |
| 17 | 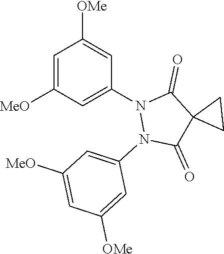 [SMILES: COc1cc(OC)cc(N2C(=O)C3(CC3)C(=O)N2c2cc(OC)cc(OC)c2)c1] | ¹H NMR (400 MHz, CDCl₃) δ 6.57 (d, J = 2.3 Hz, 4H), 6.30 (t, J = 2.2 Hz, 2H), 3.73 (s, 12H), 1.90 (s, 4H); ¹³C NMR (100 MHz, CDCl₃) δ 171.3, 160.9, 138.3, 100.8, 98.8, 55.5, 27.0, 22.0; HRMS (ESI- TOF) m/z: [M + H]⁺ calcd for C₂₁H₂₃O₆N₂ 399.1551, found 399.1555. |
| 18 | 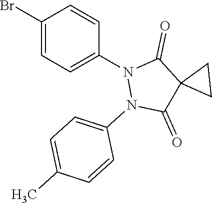 [SMILES: Cc1ccc(N2C(=O)C3(CC3)C(=O)N2c2ccc(Br)cc2)cc1] | ¹H NMR (400 MHz, CDCl₃) δ 7.45 (d, J = 8.5 Hz, 2H), 7.36-7.06 (m, 6H), 2.31 (s, 3H), 1.92 (s, 4H); ¹³C NMR (100 MHz, CDCl₃) δ 171.3, 171.0, 136.8, 135.3, 133.8, 132.0, 129.7, 123.7, 122.4, 119.7, 26.8, 21.9, 21.0; HRMS (ESI-TOF) m/z: [M + H]⁺ calcd for C₁₈H₁₆BrN₂O₂, 371.0390 found 371.0392. |
| 19 | 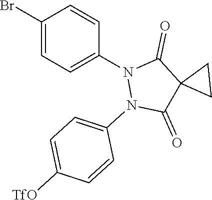 [SMILES: O=C1N(c2ccc(Br)cc2)N(c2ccc(OS(=O)(=O)C(F)(F)F)cc2)C(=O)C12CC2] | ¹H NMR (400 MHz, CDCl₃) δ 7.52-7.41 (m, 4H), 7.35-7.09 (m, 4H), 1.96 (s, 4H); ¹³C NMR (100 MHz, CDCl₃) δ 171.2, 171.1, 146.9, 136.1, 135.4, 132.3, 123.3, 123.0, 122.1, 120.2, 118.6 (q, J = 321.2 Hz, CF₃), 26.8, 22.7; HRMS (ESI-TOF) m/z: [M + H]⁺ calcd for C₁₈H₁₃BrF₃N₂O₅S 504.9675, found 504.9693. |
| 20 | 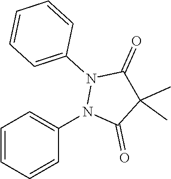 [SMILES: CC1(C)C(=O)N(c2ccccc2)N(c2ccccc2)C1=O] | ¹H NMR (200 MHz, CDCl₃) δ 7.37-7.30 (m, 8H), 7.23-7.16 (m, 2H), 1.52 (s, 6H); GC-MS m/z: [M]⁺ calcd for C₁₇H₁₆N₂O₂ 280.3, found 280.3. |
| 21 | 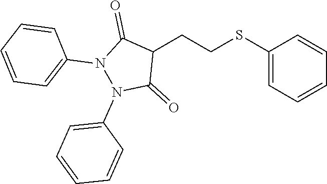 [SMILES: O=C1C(CCSc2ccccc2)C(=O)N(c2ccccc2)N1c1ccccc1] | ¹H NMR (400 MHz, CDCl₃) δ 7.36-7.23 (m, 12H), 7.23-7.11 (m, 3H), 3.64 (t, J = 6.3 Hz, 1H), 3.22 (t, J = 7.1 Hz, 2H), 2.37 (q, J = 6.7 Hz, 2H); ¹³C NMR (100 MHz, CDCl₃) δ 169.6, 135.7, 134.7, 129.9, 129.0, 128.9, 126.8, 126.5, 122.6, 44.4, 30.3, 27.0; HRMS (ESI-TOF) m/z: [M + H]⁺ calcd for C₂₃H₂₁O₂N₂S 389.1318, found 389.1315. |

### Example 9: Preparation of Sulfinpyrazone Drug

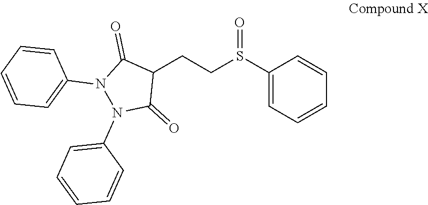 [SMILES: O=C1C(CCS(=O)c2ccccc2)C(=O)N(c2ccccc2)N1c1ccccc1]

### Step-1: Preparation of Sulfide Compound

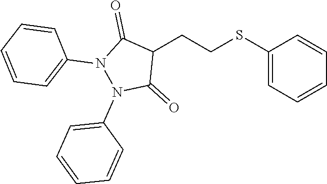 [SMILES: O=C1C(CCSc2ccccc2)C(=O)N(c2ccccc2)N1c1ccccc1]

An oven dried two-neck round bottom flask was charged with sodium methoxide (14.58 mg, 0.27 mmol, 1.5 equiv.) under the argon atmosphere and flushed with argon gas. Dry methanol (1.8 ml, 0.1 M) followed by the thiophenol (19.8 mg, 0.18 mmol, 1 equiv.) was added and the reaction mixture was kept for 30 min at room temperature before adding the example 1 (50 mg, 0.18 mmol, 1 equiv.). After the completion of the reaction (monitored by TLC, approx. 2 hrs), the solvent was evaporated and diluted with water. The aqueous part was extracted with EtOAc (3×5 ml) and the combined organic layers were dried over Na2SO4 and concentrated under reduced pressure. The resulting crude mixture was purified using flash column chromatography (20 to 50% EtOAc/Pet ether) to provide the title compound.

¹H NMR (400 MHz, CDCl₃) δ 7.36-7.23 (m, 12H), 7.23-7.11 (m, 3H), 3.64 (t, J=6.3 Hz, 1H), 3.22 (t, J=7.1 Hz, 2H), 2.37 (q, J=6.7 Hz, 2H); ¹³C NMR (100 MHz, CDCl₃) δ 169.6, 135.7, 134.7, 129.0, 129.9, 128.9, 126.8, 126.5, 122.6, 44.4, 30.3, 27.0; HRMS (ESI-TOF) m/z: [M+H]⁺ calcd for C₂₃H₂₁O₂N₂S 389.1318, found 389.1315.

### Step 2: Preparation of Sulfinpyrazone

To the solution of sulfide compound (50 mg, 0.13 mmol, 1 equiv.) obtained above in acetonitrile (1 ml, 0.13 M), t-BuOOH (6 M solution in decane, 0.26 mmol, 2 equiv), and methanesulfonic acid (0.84 μL, 10 mol %) were added. The reaction mixture was stirred at 40° C. for overnight with a small headspace of air on the closed round bottom flask. After full conversion, the reaction mixture was diluted with ethyl acetate (5 ml) and washed with saturated solution of NaHCO₃ (2×3 ml). The combined organic layers were dried over Na2SO4 and concentrated under reduced pressure. The resulting residue was purified by column chromatography on silica gel (15% ethyl acetate/DCM) to afford the title compound as a white solid. This step 2 protocol is known in the art e.g. refer, Y. Yu, M. Yan and H. Gao, Shandong Huagong, 2016, 45, 8.

¹H NMR (400 MHz, CDCl₃) δ 7.64-7.59 (m, 2H), 7.56-7.48 (m, 3H), 7.33-7.26 (m, 8H), 7.22-7.15 (m, 2H), 6.18 (brs, 1H), 3.83-3.65 (m, 1H), 3.19-3.04 (m, 1H), 2.48-2.36 (m, 1H), 2.29-2.15 (m, 1H); ¹³C NMR (100 MHz, CDCl₃) δ 170.1, 169.9, 140.9, 135.2, 135.1, 131.4, 129.5, 129.03, 129.01, 127.17, 127.15, 124.4, 122.7, 122.6, 70.4, 47.9, 29.3; HRMS (ESI-TOF) m/z: [M+H]⁺ calcd for C₂₃H₂₁O₃N₂S 405.1267, found 405.1264.

### Advantages of the Invention

The present invention provides the process for synthesis of pyrazolidine-3, 5-dione which is less toxic, eco-friendly, cheaper, high yielding, safe and simple, has good chemical selectivity and it is easy to realize industrial production.
The present invention provides the process for synthesis of pyrazolidine-3, 5-dione using simple and easily accessible dianilide precursors and avoiding use of harmful and costly diphenylhydrazine reagent.
The present invention provides the process for preparing sulfinpyrazone which reduced the cost upto 50%.
The pyrazolidin-3,5 dione intermediate can be used for the synthesis of other drug molecules as well as their analogues.
The novel intermediates may have better biological activities.

1. A 5,6-diaryl-5,6-diazaspiro [2.4]heptane-4,7-dione compound of formula (I) 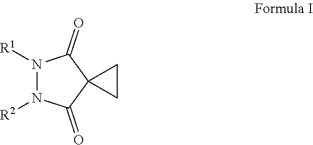
wherein
R¹ and R² may be same or different, having substituents selected from the group consisting of hydrogen, substituted or unsubstituted (C₆-C₁₀)aryl, substituted or unsubstituted (C₂-C₆)alkyl, substituted or unsubstituted heterocyclyl, —(CH₂)-substituted or unsubstituted (C₆-C₁₀)aryl, substituted or unsubstituted (C₃-C₁₀)alkene, substituted or unsubstituted (C₃-C₁₀)alkyne, ferrocene, and substituted or unsubstituted (C₁-C₆)alkoxy.

2. The compound as claimed in claim 1, wherein compound of formula (I) is selected from the group consisting of:
i. 5,6-di-p-tolyl-5,6-diazaspiro[2.4]heptane-4,7-dione;
ii. 5,6-di-m-tolyl-5,6-diazaspiro[2.4]heptane-4,7-dione;
iii. 5,6-bis(3,4-dimethylphenyl)-5,6-diazaspiro[2.4]heptane-4,7-dione;
iv. 5,6-bis(3,5-dimethylphenyl)-5,6-diazaspiro[2.4]heptane-4,7-dione;
v. 5,6-bis(4-isopropylphenyl)-5,6-diazaspiro[2.4]heptane-4,7-dione;
vi. 5,6-bis(4-(tert-butyl)phenyl)-5,6-diazaspiro[2.4]heptane-4,7-dione;
vii. 5,6-bis(4-iodophenyl)-5,6-diazaspiro[2.4]heptane-4,7-dione;
viii. 5,6-bis(4-bromophenyl)-5,6-diazaspiro[2.4]heptane-4,7-dione;
ix. 5,6-bis(4-chlorophenyl)-5,6-diazaspiro[2.4]heptane-4,7-dione;
x. 5,6-bis(3-chlorophenyl)-5,6-diazaspiro[2.4]heptane-4,7-dione;
xi. 5,6-bis(3,4-dichlorophenyl)-5,6-diazaspiro[2.4]heptane-4,7-dione;
xii. 5,6-bis(4-fluorophenyl)-5,6-diazaspiro[2.4]heptane-4,7-dione;
xiii. 5,6-bis(4-acetylphenyl)-5,6-diazaspiro[2.4]heptane-4,7-dione;
xiv. 5,6-bis(4-methoxyphenyl)-5,6-diazaspiro[2.4]heptane-4,7-dione;
xv. 5,6-Bis(4-ethoxyphenyl)-5,6-diazaspiro[2.4]heptane-4,7-dione;
xvi. 5,6-bis(3,4-dimethoxyphenyl)-5,6-diazaspiro[2.4]heptane-4,7-dione;
xvii. 5-(4-Bromophenyl)-6-(p-tolyl)-5,6-diazaspiro[2.4]heptane-4,7-dione; and
xviii. 4-(6-(4-Bromophenyl)-4,7-dioxo-5,6-diazaspiro[2.4]heptan-5-yl)phenyl trifluoromethanesulfonate.

3. A process of preparation of 5,6-diaryl-5,6-diazaspiro [2.4]heptane-4,7-dione compound of formula (I) comprising the steps of:
(a) reacting a compound of formula (3) with thionyl chloride under reflux to obtain an unstable dichloride compound of formula (3′), which was further reacted with aromatic/aliphatic amine compound of formula R¹/R²—NH₂ in presence of a base and a solvent to obtain diamide compound of formula (4); and 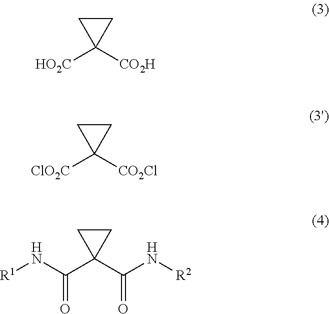
wherein R¹ and R² are the same as defined above;
(b) reacting the compound of formula (4) with an oxidant in presence of a solvent under stirring to obtain a compound of formula (I).

4. The process as claimed in claim 3, wherein the solvent used in step (a) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), 1,2-Dichloroethane (DCE), tert-Butyl alcohol (tBuOH), acetonitrile (ACN), toluene, heptane, hexane, dichloromethane (DCM), acetic acid (AcOH) or a mixture thereof.

5. The process as claimed in claim 3, wherein the base used in step (a) is selected from the group consisting of triethylamine (Et₃N), potassium carbonate, potassium bicarbonate, sodium carbonate, sodium bicarbonate, potassium hydroxide, and sodium hydroxide;

6. The process as claimed in claim 3, wherein the oxidant used in step (b) is selected from the group consisting of diacetoxyiodobenzene (PIDA), Phenyliodine bis(trifluoroacetate) (PIFA) or a mixture thereof.

7. The process as claimed in claim 3, wherein the solvent used in step (b) is selected from the group consisting of acetone, dimethylsulfoxide (DMSO), hexafluoroisopropanol (HFIP), dimethyl formamide (DMF), ethanol (EtOH), tetrahydrofuran (THF), isopropyl alcohol (IPA), methanol (MeOH), dichloroethane (DCE), tert-butyl alcohol (tBuOH), acetonitrile (ACN), toluene, heptane, hexane, dichloromethane (DCM), acetic acid (AcOH) or a mixture thereof.

8. A process of preparation of uricosuric drug sulfinpyrazone of Formula II using compound of formula I as claimed in claim 1 comprising the steps of:
(a) reacting the compound of formula (I) as claimed in claim 1, with thiophenol (PhSH) as a nucleophile in the presence of sodium methoxide solution (NaOMe) and a solvent at room temperature in the range of 20 to 35° C. for 2 hours to obtain the compound of formula (IIa)

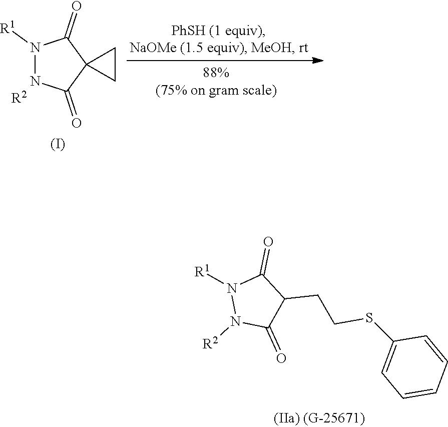

9. The process as claimed in claim 8, wherein the solvent used in step (a) is selected from the group consisting of alcoholic solvent, THF, ACN or a mixture thereof.

10. (canceled)
